(19) United States
(12) Patent Application Publication
(10) Pub. No.: US 2005/0202416 A1
(43) Pub. Date: Sep. 15, 2005

(54) Mouse hepatitis virus detection

(75) Inventors: James Horner, Methuen, MA (US); Erica Puopolo, Woburn, MA (US)

Correspondence Address: PALMER & DODGE, LLP;KATHLEEN M. WILLIAMS, 111 HUNTINGTON AVENUE, BOSTON, MA, 02199, US

(73) Assignee: Dana Farber Cancer Center

(21) Appl. No.: 10/972,860

(22) Filed: Oct. 25, 2004

Related U.S. Application Data
(63) Continuation of application No. PCT/US03/11588, filed on Apr. 16, 2003.
(60) Provisional application No. 60/375,178, filed on Apr. 24, 2002.

(51) Int. Cl. C12Q 1/70; C12Q 1/68; C07H 21/04; C12P 19/34

(52) U.S. Cl. 435/5; 435/91.2; 435/6; 536/23.72

(57) ABSTRACT

The present invention relates to the identification of a nucleotide sequence that is conserved in the genome of the Mouse Hepatitis Virus (MHV) and to the design and construction of synthetic nucleotide primers and probes that target this conserved region. The invention further provides for methods and kits that use these primers and probes to detect the presence of Mouse Hepatitis virus in a test sample.

### RELATED APPLICATIONS

This application is a continuation of PCT Application No. US03/11588, filed on Apr. 16, 2003, which claims priority to U.S. Ser. No. 60/375,178, filed on Apr. 24, 2002. The above applications are incorporated in their entirety herein by reference.

### BACKGROUND OF THE INVENTION

Mouse Hepatitis Viruses (MHV) comprise a group of approximately 25 serologically and genetically related, but distinct, strains of enveloped, single stranded RNA (+) coronaviruses that infect the laboratory mouse, Mus musculus. ‘Enterotropic’ strains replicate initially in the intestinal epithelium and usually are only weakly virulent, whereas the more virulent ‘polytropic’ strain replicates initially in the respiratory tract and tends to disseminate to the liver, brain, lymph nodes etc. Ubiquitous and highly contagious, most MHV infections of immunocompetant mice remain subclinical and clinicals symptoms, if any, are only transient (20-24 days). However, in susceptible or immunocomprimised mice and especially in newborns, MHV can cause severe acute heptatitis, encephalitis, enteritis and increased morbidity.

MHV infections therefore exhibit widely differing tissue tropism and pathogenicity which makes their early detection improbable and poses a very real threat to research results by causing immunomodulation and contamination of transplantable tumors and cell lines. These problems of MHV detection are further compounded by the high mutation rate associated with MHV RNA recombination (Rowe et al (1997) J. Virol. 71, 2959-2969; Banner and Lai, (1991) Virology 185, 441-445) and the subsequent generation of variant strains of MHV.

The consequences of MHV outbreaks can be costly. MHV infection can devastate mutant mouse colonies in a matter of weeks and recovery can take years. Currently, detection of MHV infection relies on serological assays (ELISA, Immunofluorescence assays), histopathology, electron microscopy and more recently molecular techniques (RT-PCR; Yamada et al (1993) Lan Animal Sci., 43, 285-290; Casebolt et al., (1997) Lab Animal Sci. 47, 6-10). The serological assays are, however, expensive and time consuming whereas the current RT-PCR assays are often unreliable, in part, because they lack the required sensitivity for the screening of large numbers of test samples or they fail to diagnose all MHV strains. There is therefore a need in the art for a rapid, cost effective and reliable assay for the detection and monitoring of MHV infections in laboratory mice.

### SUMMARY OF THE INVENTION

The present invention relates to the identification of a nucleotide sequence that is conserved in the genome of the Mouse Hepatitis Virus (MHV) and to the design and construction of synthetic nucleotide primers and probes that target this conserved region. The invention further provides for methods and kits that use these primers and probes to detect the presence of Mouse Hepatitis virus in a test sample.

In one embodiment, the conserved region of the Mouse Hepatitis virus genome includes at least 20 nucleotides of the DNA sequence consisting of SEQ ID NO: 1.

In a further embodiment, the synthetic nucleotide primer has the DNA sequence of SEQ ID NO:2.

In a further embodiment, the synthetic nucleotide primer has the DNA sequence of SEQ ID NO:3.

In a further embodiment, the probe has the DNA sequence of SEQ ID NO: 4. The polynucleotide probe can be a molecular beacon probe.

In one embodiment, the invention features a kit containing a pair of primers having the DNA sequences of SEQ ID NO: 2 and SEQ ID NO:3, and packaging materials therefore.

In a further embodiment, the kit further contains a probe having the DNA sequence of SEQ ID NO: 4.

In one embodiment, the invention provides a kit containing a probe having the DNA sequence of SEQ ID NO: 4 and packaging materials therefore.

In a further embodiment, the kit further includes reverse transcriptase and a nucleic acid polymerase. The nucleic acid polymerase can be thermostable.

In a further embodiment, the kit also includes a pair of reference gene-specific polynucleotide primers and a reference gene-specific probe. The probe can be a molecular beacon probe.

The present invention provides a method for detecting Mouse Hepatitis virus (MHV) in a sample by reverse transcribing RNA of a mouse sample using MHV-specific polynucleotide primers to produce MHV-specific cDNA.

In one embodiment, the invention features a method for detecting Mouse Hepatitis virus using (MHV) nucleic acid in a sample encompassing: reverse transcribing RNA of a mouse sample using MHV-specific polynucleotide primers to produce MHV-specific cDNA; and amplifying a conserved region of said MHV-specific cDNA.

In a further embodiment, the invention features a method for detecting Mouse Hepatitis virus (MHV) in a sample encompassing: reverse transcribing RNA of a mouse sample using MHV-specific polynucleotide primers to produce MHV-specific cDNA; amplifying a conserved region of said MHV-specific cDNA; and detecting the presence of said conserved region using a MHV-specific probe.

In a further embodiment, the invention features a method to detect Mouse Hepatitis virus (MHV) in a plurality of mouse samples encompassing: reverse transcribing RNA of each of a plurality of mouse samples using MHV-specific polynucleotide primers to produce a corresponding plurality of MHV-specific cDNA; amplifying a MHV conserved region of said MHV-specific cDNAs; and detecting the presence of said conserved region using a MHV-specific probe.

In one embodiment, the MHV-specific polynucleotide primers have the DNA sequence consisting of SEQ ID NO: 2 and SEQ ID NO:3.

In one embodiment, the polynucleotide primers are labeled.

In one embodiment, the conserved region comprises at least 20 nucleotides of the DNA sequence consisting of SEQ ID NO: 1.

The invention additionally provides a method encompassing the steps of: reverse transcribing RNA of a mouse sample using reference gene-specific polynucleotide primers to produce reference gene-specific cDNA; amplifying said reference gene-specific cDNA; and detecting the presence of said reference gene-specific cDNA using a reference gene-specific probe.

The invention additionally provides a method encompassing the steps of: reverse transcribing RNA of a plurality of mouse samples using reference gene-specific polynucleotide primers to produce a corresponding plurality of reference gene-specific cDNA; amplifying said reference gene-specific cDNA; and detecting the presence of said reference gene-specific cDNA using a reference gene-specific probe.

In one embodiment, the probe is a molecular beacon probe.

### DESCRIPTION OF THE DRAWINGS

FIG. 1 depicts the time of course of MHV infection as detected using the MHV Molecular Beacons Assay. The MHV molecular beacon assay was monitored using a real time PCR assay. The real-time PCR system is based on the detection and quantitation of a fluorescent reporter. This signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The numerical values depicted in the table refer to C_{T}s or the theshold cycle of a Real Time PCR assay. The threshold cycle is defined when an increase in the fluorescent signal, associated with an exponential growth of PCR product during the log-linear phase, is detected. The important parameter for quantitation is the C_{T}. The higher the initial amount of genomic DNA, the sooner accumulated product is detected in the PCR process, and the lower the C_{T} value. The choice of threshold which will determine the C_{T} value and is typically placed above any baseline activity and within the exponential increase phase. In this set of experiments the C_{T} value was set at 10 fold above background. Hence, in a positive sample, with each PCR cycle, amplification of the specific target increases. Thus, more of the labeled probe hybridizes and fluorescence increases. When the fluorescence for a sample is 10 fold above background this is the C_{T} for the sample. The more starting copies of the target sequence the sooner it will reach 10 fold above background fluorescence value and a lower C_{T} value. It is generally accepted in the field of Real-Time QPCR that a C_{T} value of 40 or 50 is equivalent to no amplification (the sample is negative).

Two chronically MHV infected ICR mice were co-housed with one SCID and one naïve immunocompetent mouse for twenty-four days. At the end of this period serum was collected from the immunocompetent mouse and screened by ELISA for MHV. The test indicated that the mouse was MHV positive and based on this result the SCID mouse was assumed to be MHV positive. At the zero time point, two naive SCID mice were co-housed with the presumed MHV positive SCID. The MHV molecular beacons assay was then run on fecal RNA isolated from mice at different time points (24, 48.72 and 120 hours) after exposure of SCID mice to an MHV infected mouse. RNA was isolated using Qiagen RNeasy kit according to manufacturers instructions. The table demonstrates that the assay is specific. The positive RNA sample amplified C_{T}˜25-28 and all negative controls failed to amplify (a CT value of 40 in experiment 1 and 50 in experiment 2). Also, RNA isolated from fecal samples at 24 and 48 hours failed to amplify (a C_{T} value of 40 in experiment 1 and 50 in experiment 2). The data indicates that at 72 hours, MHV was detected in the feces of the previously naïve SCID mouse. This denotes the earliest time point that MHV viral exposure can be detected by this assay.

### DETAILED DESCRIPTION

Definitions

As used herein, “Mouse Hepatitis Virus” (MHV) refers to enveloped, positive sense RNA mouse coronaviruses (reviewed by Homberger F R, Lab Anim 1997 31: 97-115). There are many different MHV strains that vary in virulence, organotropism and cell tropism, and are constantly evolving by naturally occurring mutation and recombination. Ubiquitous and highly contagious, MHVs typically infect the respiratory (respiratory tropic) or gastrointestinal tract (entrotropic) and cause a wide variety of diseases such as hepatitis, enteritis and encephalomyelitis. The severity of the disease depends on the strain, age and immune status of the infected mouse.

The term “molecular beacon” as used herein is single-stranded polynucleotide probes that possess a stem-and-loop hairpin structure. The loop portion of the molecule is a probe sequence complementary to a target sequence (e.g., an internal region of a sequence amplified by PCR) and the stem is formed by short complementary sequences located at the opposite ends of the molecule. The molecule is labeled with a fluorophore at one end and a quencher at the other end. When free in solution, the stem keeps the fluorophore and the quencher in close proximity, causing the fluorescence of the fluorophore to be quenched by energy transfer. When bound to its complementary target, the probe-target hybrid forces the stem to unwind, separating the fluorophore from the quencher, and restoring the fluorescence. The hairpin stem significantly enhances the specificity of molecular beacons, enabling them to distinguish targets that differ by as little as a single nucleotide. In addition, the hairpin conformation allows a variety of fluorophores to be used in conjunction with the same quencher. Thus, more than one molecular beacon, each labeled with a different fluorophore, can be used to detect several different target sequences present in the same solution.

As used herein, the term “cDNA” (complementary DNA) refers to DNA that has been derived from messenger RNA (mRNA). As known in the art, cDNA is typically generated by first reverse transcribing a first strand of cDNA from a template mRNA using a RNA dependent DNA polymerase.

As used herein, the term “amplifying”, when applied to a nucleic acid sequence, refers to a process whereby one or more copies of a particular nucleic acid sequence is generated from a template nucleic acid, preferably by the method of polymerase chain reaction (Mullis and Faloona, 1987, Methods Enzymol., 155:335). “Polymerase chain reaction” or “PCR” refers to an in vitro method for amplifying a specific nucleic acid template sequence. The PCR reaction involves a repetitive series of temperature cycles and is typically performed in a volume of 50-100 μl. The reaction mix comprises dNTPs (each of the four deoxynucleotides dATP, dCTP, dGTP, and dTTP), primers, buffers, DNA polymerase, and nucleic acid template. The PCR reaction comprises providing a set of polynucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of polynucleotide primers” or “a set of PCR primers” can comprise two, three, four or more primers. In one embodiment, an exo-Pfu DNA polymerase is used to amplify a nucleic acid template in PCR reaction.

Other methods of amplification include, but are not limited to, ligase chain reaction (LCR), polynucleotide-specific based amplification (NSBA), or any other method known in the art.

As used herein, the term “reverse transcription” or “reverse transcribing” refers to the primer-dependent process in which RNA-dependent DNA polymerase (reverse transcriptase or RT) synthesizes a single strand cDNA that is complementary to a RNA template. “Reversed transcriptase” refers to the family of enzymes that are required for the synthesis of cDNA from viral RNA for all retroviruses, including HIV, HTLV-I, HTLV-II, FeLV, FIV, SIV, AMV, MMTV, and MoMuLV (for a review, see e.g. Levin, 1997, Cell, 88:5-8; Brosius et al., 1995, Virus Genes 11:163-79).

As used herein, “polynucleotide primer” refers to a DNA or RNA molecule capable of hybridizing to a nucleic acid template and acting as a substrate for enzymatic synthesis under conditions in which synthesis of a primer extension product which is complementary to a nucleic acid template is catalyzed to produce a primer extension product which is complementary to the target nucleic acid template. The conditions for initiation and extension include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.) and at a suitable temperature. The primer is preferably single-stranded for maximum efficiency in amplification. “Primers” useful in the present invention are generally between about 10 and 35 nucleotides in length, preferably between about 15 and 30 nucleotides in length, and most preferably between about 18 and 25 nucleotides in length.

As used herein, “labeled” refers to any atom or molecule which can be used to provide a detectable (preferably quantifiable) signal, and which can be operatively linked to a polynucleotide. Labels may provide signals detectable by fluorescence, radioactivity, colorimetry, gravimetry, X-ray diffraction or absorption, magnetism, enzymatic activity, mass spectrometry, binding affinity, hybridization radiofrequency, nanocrystals and the like. A primer of the present invention may be labeled so that the amplification reaction product may be “detected” by “detecting” the detectable label. “Qualitative or quantitative” detection refers to visual or automated assessments based upon the magnitude (strength) or number of signals generated by the label. A labeled polynucleotide (e.g., a polynucleotide primer) according to the methods of the invention is labeled at the 5′ end, the 3′ end, or both ends, or internally. The label can be “direct”, e.g., a dye, or “indirect”. e.g., biotin, digoxin, alkaline phosphatase (AP), horse radish peroxidase (HRP). For detection of “indirect labels” it is necessary to add additional components such as labeled antibodies, or enzyme substrates to visualize the, captured, released, labeled polynucleotide fragment. In a preferred embodiment, an polynucleotide primer is labeled with a fluorescent label. Suitable fluorescent labels include fluorochromes such as fluorescein isocyanate (FIC), fluorescein isothiocyanate (FITC), 5-dimethylamine-1-naphthalenesulfonyl chloride (DANSC), tetramethylrhodamine isothiocyanate (TRITC), lissamine, rhodamine 8200 sulphonyl chloride (R 8200 SC) and the like (see for example, DeLuca, Immunofluorescence Analysis, in Antibody As a Tool, Marchalonis, et al., eds., John Wiley & Sons, Ltd., (1982), which is incorporated herein by reference).

As used herein, a “polynucleotide” generally refers to any polyribonucleotide or poly-deoxyribonucleotide, which may be unmodified RNA or DNA or modified RNA or DNA. “Polynucleotides” include, without limitation, single- and double-stranded polynucleotides. As used herein, the term “polynucleotide(s)” also includes DNAs or RNAs as described above, that contain one or more modified bases. Thus, DNAs or RNAs with backbones modified for stability or for other reasons are “polynucleotides”. A polynucleotide useful for the present invention may be an isolated or purified polynucleotide or it may be an amplified polynucleotide in an amplification reaction.

As used herein, “conserved region” or “conserved sequence” refers to a part of the Mouse Hepatitis virus genome which has a nucleic acid sequence that is invariant when compared to the corresponding sequences in all other isolated strains of MHV as obtainable in Genbank or similar database. In a preferred embodiment, the “conserved” region is a portion of the conserved sequence that is about 20 to 73 nucleotides of SEQ ID NO:1. In a most preferred embodiment, the conserved region according to the invention is the complete 73 nucleotide DNA sequence of SEQ ID NO: 1.

As used herein, “detecting” refers to determining the presence of an amplification reaction product by any method known to those of skill in the art, or taught in numerous texts and laboratory manuals (see, for example, Ausubel et. al. Short Protocols in Molecular Biology (1995)3^{rd} Ed. John Wiley & Sons, Inc.). In a preferred embodiment of the invention, molecular beacons are used to detect the amplification reaction products in real time during an amplification reaction (see, for example Piatek et al., 1998 Nature Biotech., 16: 359). Typically using this procedure, as few as 1-10 copies of Mouse House Hepatitis virus nucleic acid can be detected in a test sample.

Techniques which may also be used to “detect” the presence of an amplification product include, but are not limited to agarose or polyacrylamide gel electrophoresis, chromatography, Southern blot, and spectrophotometry. For example, following mixing of a MHV cDNA and polynucleotide primers, useful in the present invention, and incubation under conditions which allow for amplification of the MHV cDNA template, the amplification reaction mixture may be analyzed by agarose gel electrophoresis and ethidium bromide staining. Visualization of a stained band of the appropriate size is indicative of the presence of an amplification reaction product, and thus, the product is “detected”. The amount of amplified genomic DNA that can be reliably “detected” so as to identify a selected species using visualization of an ethidium bromide stained band which is 50 bp to 15 kb in length is at least 1 ng, such as 100 ng or 1 μg or more. Alternatively, the presence of an amplification reaction product may be determined by spectrophotometry, wherein the concentration of nucleic acid is measured following amplification of a MHV cDNA sample, and wherein an increase in the concentration of nucleic acid following amplification is an indication of the presence of a reaction product. An increase in nucleic acid concentration can therefore serve as a parameter by which an amplification reaction product is “detected”.

Alternatively, primers of the present invention may be labeled with a detectable label such as a radioactive moiety, or a fluorescent label, or alternatively, the amplification reaction may incorporate labeled nucleotides into the reaction product. Thus, the amplification reaction product may be “detected” by “detecting” the fluorescent or radioactive label, such as by autoradiography.

As used herein, the term “sample” or “samples” refers to a biological material, which is isolated from its natural environment and may contain a MHV polynucleotide. A “sample” according to the invention may consist of purified or isolated polynucleotide, or it may comprise a biological sample such as a tissue sample, a biological fluid sample, or a cell sample encompassing a MHV polynucleotide. A biological fluid includes blood, plasma, sputum, urine, cerebrospinal fluid, ravages, and leukophoresis samples. The term “tissue” as used herein is an aggregate of cells that perform a particular function in an organism and encompasses cell lines and other sources of cellular material. In a preferred embodiment of the present invention the sample or plurality of samples consists of nucleic acids derived from samples of mouse feces.

As used herein, “plurality” refers to more than two. Plurality, according to the invention, can be 3 or more, 100 or more, 1000 or more nucleic acid samples extracted from the feces of sentinel mice that can potentially contain MHV genomic nucleic acid.

A “portion”, as it is used herein, refers to a part of a conserved sequence and is typically a contiguous nucleic acid sequence of 10 nucleotides, 20 nucleotides, 30 nucleotides, 40 nucleotides, 50 nucleotides, 60 nucleotides 70 nucleotides or 73 nucleotides anywhere within the conserved sequence, as defined herein.

As used herein, “probe” refers to a molecule that binds to a specific sequence or subsequence or other moiety of another molecule. Unless otherwise indicated, the term “probe” typically refers to a polynucleotide probe that binds to another polynucleotide, often called the “target nucleic acid”, through complementary base pairing with a degree of complementarity sufficient to permit detection of the target nucleic acid. Probes may bind target nucleic acids lacking complete sequence complementarity with the probe, depending upon the stringency of the hybridization conditions. A probe according to the invention is about 20 to 73 nucleotides in length. The probe may be single or double stranded. In a preferred embodiment, the probe refers to a molecular beacon probe.

As used herein, a “primer” is a polynucleotide that is used for priming DNA synthesis by annealing to a template strand of nucleic acid through complementary base-pairing, and ranges in length from about 10 to about 50 nucleotides in length.

As used herein, “nucleic acid polymerase” refers to an enzyme that catalyzes the polymerization of nucleotides. Generally, the enzyme will initiate synthesis at the 3′-end of the primer annealed to a nucleic acid template sequence, and will proceed in the 5′-direction along the template. “DNA polymerase” catalyzes the polymerization of deoxynucleotides. Known DNA polymerases include, for example, Pyrococcus furiosus (Pfu) DNA polymerase, E. coli DNA polymerase I, T7 DNA polymerase, Thermus thermophilus (Tth) DNA polymerase, Bacillus stearothermophilus DNA polymerase, Thermococcus litoralis (Tli) DNA polymerase (also referred to as Vent DNA polymerase), Thermotoga maritima (UlTma) DNA polymerase, Thermus aquaticus (Taq) DNA polymerase, and Pyrococcus GB-D (PGB-D) DNA polymerase. The polymerase activity of any of the above enzyme can be defined by means well known in the art. One unit of DNA polymerase activity, according to the subject invention, is defined as the amount of enzyme which catalyzes the incorporation of 10 nmoles of total dNTPs into polymeric form in 30 minutes at 72° C.

As used herein, “thermostable” refers to an enzyme which is stable and active at temperatures as great as preferably between about 90-100° C. and more preferably between about 70-98° C. as compared to a non-thermostable form of an enzyme with a similar activity that are typically denatured at such elevated temperatures. For example, a representative thermostable nucleic acid polymerase isolated from Thermus aquaticus (Taq) is described in U.S. Pat. No. 4,889,818 and a method for using it in conventional PCR is described in Saiki et al., 1988, Science 239:487. Another representative thermostable nucleic acid polymerase isolated from P. furiosus (Pfu) is described in Lundberg et al., 1991, Gene, 108:1-6. Additional representative temperature stable polymerases include, e.g., polymerases extracted from the thermophilic bacteria Thermus flavus, Thermus ruber, Thermus thermophilus, Bacillus stearothermophilus (which has a somewhat lower temperature optimum than the others listed), Thermus lacteus, Thermus rubens, Thermotoga maritima, or from thermophilic archaea Thermococcus litoralis, and Methanothermus fervidus.

As used herein, “generating a signal” refers to detecting and or measuring the amplified MHV-specific cDNA as an indication of the presence of a MHV in a sample.

As used herein, “MHV-specific” refers to a nucleotide sequence that is found specifically within the genome of a Mouse Hepatitis Virus genome and not within any other viral or non viral genome. In a preferred embodiment of the present invention, “MHV-specific” refers to the conserved region of the MHV genome, as defined herein, that is specific to the Mouse Hepatitis genome and whose nucleotide sequence is invariant amongst all strains of MHV.

As used herein, “reference gene” refers to genes, typically housekeeping genes, whose expression is expected to be constant from sample to sample and therefore acts as an internal control thus permitting direct comparison between different samples. Examples of housekeeping genes typically used as internal controls in the art include GADPH and β-actin amongst others.

### DESCRIPTION

The subject invention provides for a region of the Mouse Hepatitis Virus genome that is conserved amongst all known MHV strains. The invention also features primers and probes to this conserved region and methods for their use in the detection of Mouse Hepatitis virus in a test sample.

The practice of the present invention will employ, unless otherwise indicated, conventional techniques of molecular biology, microbiology and recombinant DNA techniques, which are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Harnes & S. J. Higgins, eds., 1984); A Practical Guide to Molecular Cloning (B. Perbal, 1984); and a series, Methods in Enzymology (Academic Press, Inc.); Short Protocols In Molecular Biology, (Ausubel et al., ed., 1995). All patents, patent applications, and publications mentioned herein, both supra and infra, are hereby incorporated by reference.

A. Nucleic Acid Sequences Useful in the Invention

1. MHV Sequences According to the Invention

Mouse Hepatitis Virus Genome Sequences.

The complete 31357 bp nucleotide sequence of the Mouse Hepatitis virus has been determined and is readily accessible through Genbank (Genbank Accession Number: NC_{—}001846; Altered pathogenesis of a mutant of the murine coronavirus MHV-A59 is associated with a Q159L amino acid substitution in the spike protein Leparc-Goffart, I., Hingley, S. T., Chua, M. M., Jiang, X., Lavi, E. and Weiss, S. R. Virology 239 (1), 1-10 (1997); Virus-encoded proteinases and proteolytic processing in the Nidovirales Ziebuhr, J., Snijder, E. J. and Gorbalenya, A. E. J. Gen. Virol. 81 Pt 4, 853-879 (2000); Direct Submission to Genbank by Weiss, S. R., Leparc-Goffart, I. and Hingley, S. T. Submitted Microbiology, Univ. of Pennsylvania, 203A Johnson Pavilion, Philadelphia, Pa. 19104-6076, USA).

A MHV-Specific ‘Conserved Region’ According to the Invention

In a preferred embodiment, the invention provides for a polynucleotide sequence of SEQ ID NO: 1. This 73 nucleotide sequence, which is located in the 3′ untranslated region downstream of the nucleocapsid open reading frame, is located between nucleotides 31043 and 31150 of the MHV genomic sequence as reported in Genbank (Accession number NC 001846). A BLAST search using SEQ ID NO:1 demonstrated 100% identity with all known strains of MHV including, but not limited to, strains ML-11, ML-10, MHV-A59, Penn 97-1, JHM, MHV strains 1 and 2.

2. Primers and Probes According to the Invention

The invention provides for oligonucleotide primers and probes useful for detecting or measuring the presence of MHV conserved sequence of SEQ ID NO:1 and for amplifying MHV-specific sequences of SEQ ID NO:1 in different strains of MHV.

a. MHV-Specific Nucleotide Primers

Primer Design

Primers may be selected manually by analyzing the conserved region of MHV of SEQ ID NO: 1. Computer programs, however, are also available in selecting primers to generate an amplified product with a designed length, e.g., primer premier 5 (www.premierbiosoft.com) and primer3 (www-genome.wi.mit.edu).

It is known in the art that primers that are about 20-25 bases long and with 50% G-C content will work well at annealing temperature at about 52-58° C. These properties are preferred when designing primers for the subject invention. Longer primers, or primers with higher G-C contents, have annealing optimums at higher temperatures; similarly, shorter primers, or primers with lower G-C contents, have optimal annealing properties at lower temperatures. A convenient, simplified formula for obtaining a rough estimate of the melting temperature of a primer 17-25 bases long is as follows:
Melting temperature (Tm in ° C.)=4×(# of G+# of C)+2×(# of A+# of T)

Shorter fragments are amplified more efficiently than longer fragments although target of more than 10 kb can be successfully amplified. Therefore preferably primers are selected so to amplify a relatively short product. Preferably, primers are selected to generate an amplified product of less than 500 bp, or 200 bp, or more preferably 100 bp in length or most preferably 73 bp in length.

In accordance with the preferred embodiments, optimal results have been obtained using primers, which are 19-25 in length. For the amplification of the conserved region of DNA sequence SEQ ID NO:1, primers were selected having the DNA sequence of SEQ ID NO: 2 and SEQ ID NO: 3. However, one skilled in the art will recognize that the length of the primers used may vary. For example, it is envisioned that shorter primers containing at least 15, and preferably at least 17, consecutive bases of the nucleotide sequences of these primers of SEQ ID NO: 2 and SEQ ID NO: 3 may be suitable. The exact upper limit of the length of the primers is not critical. However, typically the primers will be less than or equal to approximately 50 bases, preferably less than or equal to 30 bases. Further still, the bases included in the primers may be modified as is conventional in the art, including but not limited to, incorporating detectable labels such as biotin, or fluorescent labels.

Primer Synthesis

Methods for synthesizing primers are available in the art. The oligonucleotide primers of this invention may be prepared using any conventional DNA synthesis method, such as, phosphotriester methods such as described by Narang et al. (1979, Meth. Enzymol., 68:90) or Itakura (U.S. Pat. No. 4,356,270), or and phosphodiester methods such as described by Brown et al. (1979, Meth. Enzymol., 68:109), or automated embodiments thereof, as described by Mullis et al. (U.S. Pat. No. 4,683,202). Also see particularly Sambrook et al.(1989), Molecular Cloning: A Laboratory Manual (2d ed.; Cold Spring Harbor Laboratory: Plainview, N.Y.), herein incorporated by reference.

The primers, according to the subject invention, may be incorporated into a convenient kit for MHV-specific amplification and detection.

Labeling of the Polynucleotide Primers

The polynucleotide primer of the present invention may be labeled, as described below, by incorporating moieties detectable by spectroscopic, photochemical, biochemical, immunochemical, enzymatic or chemical means. The method of linking or conjugating the label to the oligonucleotide primer depends, of course, on the type of label(s) used and the position of the label on the primer. A primer that is useful according to the invention can be labeled at the 5′ end, the 3′ end or labeled throughout the length of the primer.

A variety of labels that would be appropriate for use in the invention, as well as methods for their inclusion in the primer, are known in the art and include, but are not limited to, enzymes (e.g., alkaline phosphatase and horseradish peroxidase) and enzyme substrates, radioactive atoms, fluorescent dyes, chromophores, chemiluminescent labels, electrochemiluminescent labels, such as Origen™ (Igen), that may interact with each other to enhance, alter, or diminish a signal. Of course, if a labeled molecule is used in a PCR based assay carried out using a thermal cycler instrument, the label must be able to survive the temperature cycling required in this automated process.

Fluorophores for use as labels in constructing labeled primers of the invention include rhodamine and derivatives (such as Texas Red), fluorescein and derivatives (such as 5-bromomethyl fluorescein), Lucifer Yellow, IAEDANS, 7-Me₂N-coumarin-4-acetate, 7-OH-4-CH₃-coumarin-3-acetate, 7-NH₂-4-CH₃-coumarin-3-acetate (AMCA), monobromobimane, pyrene trisulfonates, such as Cascade Blue, and monobromorimethyl-ammoniobimane. In general, fluorophores with wide Stokes shifts are preferred, to allow using fluorimeters with filters rather than a monochromometer and to increase the efficiency of detection.

The labels may be attached to the polynucleotide directly or indirectly by a variety of techniques. Depending on the precise type of label or tag used, the label can be located at the 5′ or 3′ end of the primer, located internally in the primer, or attached to spacer arms of various sizes and compositions to facilitate signal interactions. Using commercially available phosphoramidite reagents, one can produce oligomers containing functional groups (e.g., thiols or primary amines) at either the 5- or the 3-terminus via an appropriately protected phosphoramidite, and can label them using protocols described in, for example, PCR Protocols: A Guide to Methods and Applications, Innis et al., eds. Academic Press, Ind., 1990.

Methods for introducing polynucleotide functionalizing reagents to introduce one or more sulfhydryl, amino or hydioxyl moieties into the oligonucleotide primer sequence, typically at the 5′ terminus, are described in U.S. Pat. No. 4,914,210. A 5′ phosphate group can be introduced as a radioisotope by using polynucleotide kinase and gamma-³²P-ATP or gamma-³³P-ATP to provide a reporter group. Biotin can be added to the 5′ end by reacting an aminothymidine residue, or a 6-amino hexyl residue, introduced during synthesis, with an N-hydroxysuccinimide ester of biotin. Labels at the 3′ terminus may employ polynucleotide terminal transferase to add the desired moiety, such as for example, cordycepin ³⁵S-dATP, and biotinylated dUTP.

b. MHV-Specific Probes

Probes According to the Invention

The “MHV-specific probe”, which is typically from 20-73 nucleotides in length, comprises a nucleic acid sequence that is complementary to the corresponding nucleotides of the amplified MHV conserved region of SEQ ID NO: 1 or a portion thereof and is capable of selectively hybridizing to these sequences. Selective hybridization occurs when two nucleic acid sequences are substantially complementary (at least about 95% complementary over a stretch of at least 14 to 25 nucleotides, preferably at least about 99%, more preferably at least about 100% complementary). See Kanehisa, M., 1984, Nucleic Acids Res. 12: 203, incorporated herein by reference. As a result, it is expected that a certain degree of mismatch at the priming site is tolerated. Such mismatch may be small, such as a mono-, di- or tri-nucleotide. Alternatively, a region of mismatch may encompass loops, which are defined as regions in which there exists a mismatch in an uninterrupted series of four or more nucleotides. In a preferred embodiment of the invention, the probe has 100% complementarity with the target MHV conserved sequence and does not contain a sequence complementary to sequence(s) other than MHV conserved sequences.

Numerous factors influence the efficiency and selectivity of hybridization of the probe to a targeted MHV conserved sequences. These factors, which include probe length, nucleotide sequence and/or composition, hybridization temperature, buffer composition and potential for steric hindrance in the region to which the primer is required to hybridize, will be considered when designing probes according to the invention.

In a preferred embodiment, a MHV-specific probe, according to the invention, refers to a probe having a secondary structure that changes upon binding of the probe to a target nucleic acid within the conserved region of SEQ ID NO: 1. The MHV-specific probe comprises a binding moiety, wherein the probe forms a duplex structure with a sequence in the target nucleic acid, due to complementarity of at least one sequence in the probe with a sequence in the target region. A probe according to the invention can also be end labeled or internally labelled using a number of means well known to those of skill in the art.

Molecular Beacon Probes

In a most preferred embodiment, the probe, according to the invention, is a molecular beacon probe. Molecular beacon probes comprise a hairpin, or stem-loop structure which possesses a pair of interactive signal generating labeled moieties (e.g., a fluorophore and a quencher) effectively positioned to quench the generation of a detectable signal when the beacon probe is not hybridized to the MHV target nucleic acid. The loop comprises a region that is complementary to a MHV target nucleic acid. The loop is flanked by 5′ and 3′ regions (“arms”) that reversibly interact with one another by means of complementary nucleic acid sequences when the region of the probe that is complementary to a nucleic acid target sequence is not bound to the target nucleic acid. Alternatively, the loop is flanked by 5′ and 3′ regions (“arms”) that reversibly interact with one another by means of attached members of an affinity pair to form a secondary structure when the region of the probe that is complementary to a nucleic acid target sequence is not bound to the target nucleic acid. As used herein, “arms” refers to regions of a molecular beacon probe that a) reversibly interact with one another by means of complementary nucleic acid sequences when the region of the probe that is complementary to a nucleic acid target sequence is not bound to the target nucleic acid or b) regions of a probe that reversibly interact with one another by means of attached members of an affinity pair to form a secondary structure when the region of the probe that is complementary to a nucleic acid target sequence is not bound to the target nucleic acid. When a molecular beacon probe is not hybridized to a target, the arms hybridize with one another to form a stem hybrid, which is sometimes referred to as the “stem duplex”. This is the closed conformation. When a molecular beacon probe hybridizes to its target the “arms” of the probe are separated. This is the open conformation. In the open conformation an arm may also hybridize to the target. Such probes may be free in solution, or they may be tethered to a solid surface. When the arms are hybridized (e.g., form a stem) the quencher is very close to the fluorophore and effectively quenches or suppresses its fluorescence, rendering the probe dark. Such probes are described in U.S. Pat. No. 5,925,517 and U.S. Pat. No. 6,037,130. The invention encompasses molecular beacon probes wherein one or more subunits of the probe comprise a molecular beacon structure.

Molecular beacon probes have a fluorophore attached to one arm and a quencher attached to the other arm. The fluorophore and quencher, for example, tetramethylrhodamine and DABCYL, need not be a FRET pair. For example, in one embodiment, a fluorophore is attached to one arm of the probe subunit encompassing a molecular beacon structure and a quencher is attached to the other arm of the probe subunit encompassing a molecular beacon structure.

For stem loop probes useful in this invention, the length of the probe or probe subunit sequence that is complementary to the target, the length of the regions of a probe or probe subunit (e.g., stem hybrid) that reversibly interact with one another by means of complementary nucleic acid sequences, when the region complementary to a nucleic acid target sequence is not bound to the target nucleic acid, and the relation of the two, is designed according to the assay conditions for which the probe is to be utilized. The lengths of the target-complementary sequences and the stem hybrid sequences for particular assay conditions can be estimated according to what is known in the art. The regions of a probe that reversibly interact with one another by means of complementary nucleic acid sequences when the region of the probe that is complementary to a nucleic acid target sequence is not bound to the target nucleic acid are in the range of 6 to 100, preferably 8 to 50 nucleotides and most preferably 8 to 25 nucleotides each. The length of the probe sequence that is complementary to the target is preferably 17-40 nucleotides, more preferably 17-30 nucleotides and most preferably 17-25 nucleotides long.

In a preferred embodiment of the invention, primer and molecular beacon probe sets used to target conserved sequences within the MHV genome are selected using Primer Express software (Perkin Elmer/Applied Biosystems). Whenever possible, primers and probes should be selected in a region with a G/C content of 20-80%. Regions with a G/C content in excess of this may not denature well during thermal cycling, leading to a less efficient reaction. In addition, G/C-rich sequences are susceptible to non-specific interactions that may reduce reaction efficiency and produce non-specific signals. For this same reason, primer and probe sequences containing runs of four or more G bases should be avoided. A/T-rich sequences require longer primer and probe sequences in order to obtain the recommended Tms. This is rarely a problem for quantitative assays; however, probes approaching 40 base pairs can exhibit less efficient quenching and produce lower synthesis yields. Selecting primers and probes with the recommended Tms is one of the factors that allows the use of universal thermal cycling parameters. Having the probe Tm 8-10° C. higher than that of the primers ensures that the probe is fully hybridized during primer extension.

In a preferred embodiment of the instant invention, the molecular beacon probe has the DNA sequence of SEQ ID NO: 4.

The probe, according to the subject invention, may be incorporated into a convenient kit for MHV-specific amplification and detection.

Labeling of Probes According to the Invention

The probe, according to the present invention, can also be labeled via operative linkage of any atom or molecule which can be used to provide a detectable (preferably quantifiable) signal. Labels may provide signals detectable by fluorescence, radioactivity, colorimetry, gravimetry, X-ray diffraction or absorption, magnetism, enzymatic activity, mass spectrometry, binding affinity, hybridization radiofrequency, nanocrystals and the like. A probe of the present invention may be labeled so that the amplification reaction product may be “detected” by “detecting” the detectable label. “Qualitative or quantitative” detection refers to visual or automated assessments based upon the magnitude (strength) or number of signals generated by the label. A labeled polynucleotide (e.g., th probe) according to the methods of the invention is labeled at the 5′ end, the 3′ end, or both ends, or internally. The label can be “direct”, e.g., a dye, or “indirect”. e.g., biotin, digoxin, alkaline phosphatase (AP), horse radish peroxidase (HRP). In a preferred embodiment, a probe is labeled with a fluorescent label. Suitable fluorescent labels include fluorochromes such as fluorescein isocyanate (FIC), fluorescein isothiocyanate (FITC), 5-dimethylamine-1-naphthalenesulfonyl chloride (DANSC), tetramethylrhodamine isothiocyanate (TRITC), lissamine, rhodamine 8200 sulphonyl chloride (R 8200 SC) and the like (see for example, DeLuca, Immunofluorescence Analysis, in Antibody As a Tool, Marchalonis, et al., eds., John Wiley & Sons, Ltd., (1982), which is incorporated herein by reference).

Preparing Template for Amplification Assays

Useful templates for MHV-specific amplification, according to the invention, include biological samples taken from mice such as blood serum and tissue biopsies. In a preferred embodiment, the samples are fresh fecal samples. Total RNA is then isolated from feces using Qiagen RNeasy Micro Isolation Kit Cat. # 74103 according to manufacturer's instructions. This procedure includes a column DNase digestion step to eliminate any DNA contamination in the test samples.

B. Enzymes Useful in the Invention

Useful DNA Polymerases And Reverse Transcriptases

DNA polymerases and their properties are described in detail in, among other places, DNA Replication 2nd edition, Kornberg and Baker, W. H. Freeman, New York, N.Y. (1991).

Known conventional DNA polymerases include, for example, Pyrococcus furiosus (Pfu) DNA polymerase (Lundberg et al., 1991, Gene, 108: 1, provided by Stratagene), Pyrococcus woesei (Pwo) DNA polymerase (Hinnisdaels et al., 1996, Biotechniques, 20:186-8, provided by Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent DNA polymerase, Cariello et al., 1991, Polynucleotides Res, 19: 4193, provided by New England Biolabs), 9° Nm DNA polymerase (discontinued product from New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep-Vent DNA polymerase, Juncosa-Ginesta et al., 1994, Biotechniques, 16:820, provided by New England Biolabs), UlTma DNA polymerase (from thermophile Thermotoga maritima; Diaz and Sabino, 1998 Braz J. Med. Res, 31:1239; provided by PE Applied Biosystems), Tgo DNA polymerase (from thermococcus gorgonarius, provided by Roche Molecular Biochemicals), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DP1/DP2 DNA polymerase II (Cann et al., 1998, Proc Natl Acad Sci USA 95:14250-5). The polymerization activity of any of the above enzymes can be defined by means well known in the art. One unit of DNA polymerization activity of conventional DNA polymerase, according to the subject invention, is defined as the amount of enzyme which catalyzes the incorporation of 10 nmoles of total deoxynucleotides (dNTPs) into polymeric form in 30 minutes at optimal temperature (e.g., 72° C. for Pfu DNA polymerase). Assays for DNA polymerase activity and 3′-5′ exonuclease activity can be found in DNA Replication 2nd Ed., Kornberg and Baker, supra; Enzymes, Dixon and Webb, Academic Press, San Diego, Calif. (1979), as well as other publications available to the person of ordinary skill in the art.

When using the subject compositions in reaction mixtures that are exposed to elevated temperatures, e.g., during the PCR technique, use of thermostable DNA polymerases is preferred.

Reverse transcriptases useful according to the invention include, but are not limited to, reverse transcriptases from HIV, HTLV-1, HTLV-II, FeLV, FIV, SIV, AMV, MMTV, MoMuLV and other retroviruses (for reviews, see for example, Levin, 1997, Cell, 88:5-8; Verma, 1977, Biochim Biophys Acta. 473:1-38; Wu et al., 1975, CRC Crit Rev Biochem. 3:289-347).

Reverse transcriptase and nucleic acid polymerases, according to the subject invention, may be incorporated into a convenient kit for MHV-specific amplification and detection.

C. Methods Useful to the Invention

Assay for MHV Detection

The subject invention provides a MHV identification method that includes reverse transcribing MHV RNA in a test sample, amplifying the resulting MHV cDNA and assaying for the presence of the above-mentioned amplification products using a MHV-specific probe. The most preferred method, according to the invention are as described herein in Example 1.

MHV-Specific Reverse Transcription

The present invention provides for reverse transcription of RNA in a test sample using MHV-specific nucleotide primers. Methods of reverse transcribing RNA into cDNA are well known in the art and are described in Ausubel et al., John Wiley & Sons, Inc., 1997, Current Protocols in Molecular Biology. Alternative methods for reverse transcription utilize thermostable DNA polymerases. These methods are described in WO 90/07641.

MHV-Specific Amplification

A number of template dependent processes are available to amplify MHV-specific cDNA. One of the best known amplification methods is the polymerase chain reaction (referred to as PCR) which is described in detail in U.S. Pat. Nos. 4,683,195, 4,683,202 and 4,800,159, each of which is incorporated herein by reference in its entirety.

Another method for amplification is the ligase chain reaction (“LCR”), disclosed in European Application No. 320 308, incorporated herein by reference in its entirely. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present invention. In this method, a replicative sequence of RNA which has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention. Walker et al., Proc. Nat'l Acad. Sci. USA 89:392-396 (1992), incorporated herein by reference in its entirety.

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases may be added as biotinylated derivatives for easy detection. A similar approach is used in SDA. Target specific sequences may also be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

Still other amplification methods described in GB Application No. 2 202 328, and in PCT Application No. PCT/US89/01025, each of which is incorporated herein by reference in its entirety, may be used in accordance with the present invention. In the former application, “modified” primers are used in a PCR like, template and enzyme dependent synthesis. The primers may be modified by labelling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labelled probe signals the presence of the target sequence.

Other nucleic acid amplification procedures include transcription-based amplification systems (TAS), including nucleic acid sequence based amplification (NASBA) and 3SR. Kwoh et al., Proc. Nat'l Acad. Sci. USA 86:1173 (1989); Gingeras et al., PCT Application WO 88/10315, incorporated herein by reference in their entirety. In NASBA, the nucleic acids may be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

Davey et al., European Application No. 329 822 (incorporated herein by reference in its entirely) disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence may be used by the appropriate RNA polymerase to make many RNA copies of the DNA These copies may then re-enter the cycle leading to very swift amplification. With proper choice of enzymes, this amplification may be done isothermally without addition of enzymes at each cycle. Because of the cyclical nature of this process, the starting sequence may be chosen to be in the form of either DNA or RNA. Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR-” Frohman, M. A., In: PCR PROTOCOLS: A GUIDE TO METHODS AND APPLICATIONS, Academic Press, N.Y. (1990) and Ohara et al., Proc. Nat'l Acad. Sci. USA, 86:5673-5677 (1989), each herein incorporated by reference in their entirety. Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., Genomics 4:560 (1989), incorporated herein by reference in its entirety.

MHV-Specific PCR

Preferably, the MHV amplification, according to the invention, is carried out by PCR.

In PCR, two primer sequences are prepared which are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

Generally, PCR is conducted in a reaction mixture encompassing a suitable buffer. Any PCR buffer known in the art may be useful in the subject invention (e.g., TaqPlus Precision buffer, Stratagene, La Jolla, Cat# 600210). The reaction mixture also comprises the template DNA, the DNA polymerase, primers and an ample amount of each of the four deoxynucleoside triphosphates (dATP, dCTP, dGTP, and TTP).

The amount of polymerase must be sufficient to promote DNA synthesis throughout the predetermined number of amplification cycles. Guidelines as to the actual amount of polymerase are generally provided by the supplier of the PCR reagents and are otherwise readily determinable by a person of ordinary skill in the art. DNA polymerases useful according to the invention, include, but are not limited to, Pyrococcus furiosus (Pfu) DNA polymerase (28), E. coli DNA polymerase 1 (29), T7 DNA polymerase (30), Thermus thermophilus (Tth) DNA polymerase (31), Bacillus stearothermophilus DNA polymerase (32), Thermococcus litoralis (Tli) DNA polymerase (also referred to as Vent DNA polymerase, 33), Thermotoga maritima (UlTma) DNA polymerase (34), Thermus aquaticus (Taq) DNA polymerase (35), Pyrococcus kodakaraensis KOD DNA polymerase (36), JDF-3 DNA polymerase (37), and Pyrococcus GB-D (PGB-D) DNA polymerase (38).

The amount of each primer must be in substantial excess of the amount of target DNA to be amplified. The amount of primer needed for the reaction mixture can be estimated by one skilled in the art in terms of the ultimate number of amplified fragments desired at the conclusion of the reaction.

To prevent false positive results, one skilled in the art will recognize that the assays should include negative controls as is conventional in the art. For instance, suitable negative controls may contain no primer or no DNA (i.e. “water controls”).

Optimization of PCR Conditions

Successful MHV-specific amplification, according to the invention, depends in great measure on the specific hybridization of the MHV-specific primers to the corresponding matched template. If the primer hybridizes non-specifically to many different target sequences in the initial template, the amplification process will not be MHV-specific. The non-specific hybridization not only gives high background, but also gives false positive results and reduces the yield of desired amplification and the sensitivity of the assay.

When using a MHV-specific primer in an amplification assay, the specific choice of reagents and conditions (e.g., buffers, dNTP concentrations, and annealing temperature) need to be made to obtain maximal specificity and sensitivity (efficiency) for each template used.

Controlling the Specificity of Hybridization

The specificity of primer hybridization to template is controlled by the ionic strength (primarily the K⁺ concentration) of the buffer, the Mg²⁺ concentration (which is bound to dNTPs and therefore affected by the amount of dNTPs), and the annealing temperature of each cycle of the amplification.

In preferred embodiments, the dNTP concentrations are 50 nM, preferably 100 nM, more preferably 200 nM.

In PCR amplification, the specificity of the annealing is most important in the first few cycles. The remaining cycles only serve to expend the pool of template which is amplified in the first few cycles.

Conditions for specific hybridization of primers to particular template targets must be determined empirically, usually by varying the annealing temperature in several degree increments and comparing the specificity and sensitivity of the amplification process by agarose gel electrophoresis (See Current Protocol in Molecular Biology, supra).

The formula for calculating primer annealing temperature provided above is only a rough guide, successive trials at different annealing temperatures is the usual way to optimize this important parameter in the MHV-specific amplification reaction. Apparatus are available for simultaneous testing of different annealing temperatures of particular primer-template pairs, which enables the optimal annealing temperature to be determined rapidly and reliably (e.g., Robocycler Gradient Temperature Cycler, Cat # 400864, Stratagene; Eppendorf mastercycler gradient, Cat # 5331 000.045, Brinkmann Instruments, Inc. Westbury, N.Y.).

In some embodiments, the target sequences are amplified at an annealing and extending temperature that is between 1° C. and 10° C. higher than the Tm for the primer pair. Although amplification at this temperature is inefficient, any primer extension that occurs is target specific. Consequently, during the high temperature cycle(s), the sample is enriched for the particular target sequence and any number of cycles, i.e., 1-15 enhances product specificity. The annealing temperature may be then decreased to increase amplification efficiency and provide a detectable amount of PCR product.

Alternatively, one can simultaneously run a set of reactions at a constant temperature but vary the concentration of KCl or MgCl₂ or add variable amounts of a denaturant such as formamide (e.g., 0, 2, 4, 6%) to define the optimum conditions for generating a high yield of specific product with a minimum of nonspecific products (39, 40).

In some embodiments of the invention, Taq polymerase buffer (Stratagene, Cat # 600131) is used to provide a desirable ionic strength for MHV-specific PCR amplification.

In general, it is preferred but not essential that the DNA polymerase is added to the amplification reaction mixture after both the primer and template are added. Alternatively, for example, the enzyme and primer are added last or the reaction buffer or template plus buffer are added last. It is generally desirable that at least one component that is essential for polymerization not be present until such time as the primer and template are both present, and the enzyme can bind to and extend the desired primer/template substrate. This method, termed “hot start,” improves specificity and minimizes the formation of “primer-dimer.”

Sensitivity of Amplification

The sensitivity of the MHV-specific amplification of the subject invention depends on the template and primers used in an amplification reaction, as well as ionic strength and annealing temperature of each cycle of the amplification.

As few as one or two copies of the template (about 3-5 pg) can be used for successful PCR amplification if the reaction condition has been optimized. However, it's known in the art that a higher template concentration may increase the specificity and efficiency of the amplification.

Shorter fragments are amplified more efficiently than longer fragments. Preferably, primers which generate an amplified product of less than 500 bp in length, or preferably les than 100 bp are used to increase sensitivity of the amplification assay.

Detection of MHV-Specific Amplified Products

Direct Visualization

At the conclusion of the amplification reaction, the amplified products may be detected using techniques conventional in the art.

The cycle of DNA denaturation, primer annealing and synthesis of the DNA segment defined by the 5′ ends of the primers is repeated as many times as is necessary to amplify the template target until a sufficient amount of either a MHV-specific product is available for detection.

The primers may be labeled for facilitating the detection. The primers can be labeled with a directly detectable tag, for example a radioactive label such as ³²P, ³⁵S, ¹⁴C or ¹²⁵I, a fluorescent compound such as fluorescein or rhodamine derivatives, an enzyme such as a peroxidase or alkaline phosphatase, or avidin or biotin.

The fragments of amplified DNA are then separated according to size. This may be achieved by electrophoresis or by high pressure liquid chromatography. The separation is effected on a substrate. For electrophoresis, the substrate typically is a gel which does not denature the DNA, such as polyacrylamide gel or agarose gel.

In a preferred embodiment, the amplification products are conveniently analyzed by gel electrophoresis.

Electrophoresis is conducted under conditions which effect a desired degree of resolution of fragments. A degree of resolution that separates fragments that differ in size by as little as about 500 bp is usually sufficient. Preferably, the resolution is at about 100 bp. More preferably, the resolution is at about 10 bp. Size markers may also be run on the gel to permit estimation of the size of fragments.

The amplification product pattern may be visualized. Where an amplification primer has been labeled, this label may be revealed. A substrate carrying the separated labeled DNA fragments is contacted with a reagent which detects the presence of the label. For example, an amplified product generated from a radioactively labeled primer may be detected by radioautography. Where the amplification primers are not labeled, the substrate bearing the PCR product may be contacted with ethidium bromide and the DNA fragments visualized under ultraviolet light. In a preferred embodiment, the pattern is visualized by Eagle-Eye.

Detection Using a MHV-Specific Probe

The present invention provides for the detection of amplified MHV cDNA using a MHV-specific probe that is complementary to at least 20 nucleotides of the amplified MHV-specific cDNA sequence.

Detection methods generally employed in standard PCR techniques use a labeled probe with the amplified DNA in a hybridization assay. Preferably, the MHV-specific probe is labeled, e.g., with ³²P, biotin, horseradish peroxidase (HRP), etc., to allow for detection of hybridization. Preferably the probe is 200 nucleotides, or 100 nucleotides, or 50 nucleotides or most preferably 20 nucleotides in length. A number of standard hybridization procedures are known in the art for the detection of a target MHV nucleic acid sequence using labeled probes. In one embodiment, MHV PCR amplified cDNA is detected using a MHV-specific 20 nucleotide labeled probe, as defined herein, in a Southern hybridization assay, as described in Ausubel et al., John Wiley & Sons, Inc., 1997, Current Protocols in Molecular Biology).

A MHV-specific probe can also be an oligonucleotide with secondary structure such as a hairpin or a stem-loop, and includes, but is not limited to molecular beacons, safety pins, scorpions, and sunrise/amplifluor probes (Whitcombe et al., Nature Biotechnology 17: 804-807 (1999); Nazarneko et al., Nucleic Acid Res. 25: 2516-2521 (1997).

Detection using a MHV-Specific Molecular Beacon Probe

General Information

Molecular beacon probes are highly specific and can detect a difference of one nucleotide between the probe and the target nucleic acid sequence (Marras S A, Kramer F R, Tyagi S. Multiplex detection of single-nucleotide variations using molecular beacons. Genet Anal 1999;14(5-6):151-6; Smit M L, Giesendorf B A, Vet J A, Trijbels F J, Blom H J. Semiautomated DNA mutation analysis using a robotic workstation and molecular beacons. Clin Chem 2001; 47(4):739.44; Tyagi S, Brata D P, Kramer F R. Multicolor molecular beacons for allele discrimination. Nat Biotechnol 1998; 16(1):49-53). Two studies of Mycobacterium tuberculosis demonstrated specificity of one hundred percent (Piatek A S, Telenti A, Murray M R, El-Hajj H, Jacobs W R Jr. Tuberculosis in two distinct populations using molecular beacons; implications for rapid susceptibility testing. Antimicrob Agents Chemother 2000; 44(1):103-10; Piatek A S, Tyagi S, Pol A C, Telenti A, Miller L P, Kramer F R, Alland D. Nat Biotechnol 1998; 16(4):359-63). Molecular beacon assays also have a higher accuracy than conventional PCR (Van Schie R C, Marras S A, Conroy J M, Nowak N J, Catanese J J, dejong P J. Semiautomated clone verification by real-time PCR using molecular beacons. Biotechniques 2000; 29(6)):1296-1300, 1302-4, 1306 passim.). This high specificity is due to the hairpin structure of the probe that ensures that that fluorescence can only increase when the probe is hybridized to a specific target.

In a preferred embodiment, the probe, according to the invention, is a molecular beacon probe. In another preferred embodiment, the MHV molecular beacon probe is designed to a highly conserved region of the MHV RNA genome of SEQ ID NO: 4 and can detect all strains of MHV currently available in GenBank.

Sensitivity of the Molecular Beacon Assay

Molecular beacon assays designed to detect pathogenic human retroviruses have demonstrated a detection level of less than 10 copies of the viral genome (Vet J A, Majithia A R, Marras S A, Tyagi S, Dube S, Poiesz B J, Kramer F R. Multiplex detection of four pathogenic retroviruses using molecular beacons. Proc Natl Acad Sci 1999; 25; 96(11):6394-9.). This level of sensitivity is approximately 10 times greater than standard RT-PCR (Garin D, Peyrefitte C, Crance J M, LeFaou A, Jouan A, Bouloy M. Highly sensitive Taqman PCR detection of Poumala hanta virus. Microbes Infect 2001; 3(9):739-45). RT-PCR has demonstrated greater sensitivity than indirect immunofluorescence assay (IFA) and ELISA (Rodriquez Saint-Jean S, Borrego J J, Perez-Prieto S I. Comparative evaluation of five serological methods and RT-PCR assay for the detection of IPNV in fish. J Virol Methods 2001; 97(1-2):23-31; Li L, Wang W L, Yu X X, Zhen J Y. Detection of hepatitis C virus RNA in the tissue of hepatocellular carcinoma by multiple detection system. Zhonghua Shi Yan He Lin Chuang Bing Du Xue Za Zhi 2000; 14(1):47-52). ELISA and IFA are the current standard methods to detect MHV. Monoclonal antibody production (MAP) assay is the most sensitive serological test currently available. Standard RT-PCR was demonstrated to be equal to or greater in sensitivity to MAP screening for MHV (Homberger F R, Smith A L, Barthold S W. Detection of rodent corona viruses in tissues and cell cultures by using polymerase chain reaction. J Clin Microbiol 1991; 29(12):2789-93). MAP screening however requires approximately one month to complete.

The present invention also allows for the simultaneous use of two or more molecular beacon probes in the same reaction. The molecular beacons can differ only by a single nucleotide, therefore not only enabling the detection of MSV generally in a sample but also enabling the discrimination of the specific MSV strain within a sample. It also definitively discriminates a true negative result from a false negative result that is due to PCR failure by the use of an internal amplification control in the same reaction. Therefore, the molecular beacons of the present invention are particularly suitable as hybridization probes for MSV detection and for MSV strain discrimination.

The Molecular Beacon Assay

Molecular beacons are single-stranded oligonucleotides that possess a stem-and-loop hairpin structure. The loop is comprised of a DNA sequence complimentary to the target DNA, and the stem is formed by short complimentary sequences at opposite ends of the molecule. The ends of molecular beacons are labeled with a fluorophore and a quencher, respectively. When the molecular beacon is folded, the fluorophore and quencher are in close proximity, and fluorescence is quenched. However, when the molecular beacon is bound to a complimentary target, the fluorophore and quencher are separated, and the molecular beacon fluoresces.

Primer and probe sets to be used in the Molecular Beacon assay are designed according to guidelines obtained from computer programs such as Primer Express (PE/Applied Biosystems). The experimental procedures on the use of molecular beacons are described in Tyagi, S., et al. (1998) Nature Biotech. 16: 49-53; U.S. Pat. No. 5,925,517 and in Kostrikis, L. G., et al. (1998) Science 279: 1228-1229.

MHV Identification Using Microarray

DNA microarray techniques are well known in the art (e.g., U.S. Pat. No. 5,807,522, incorporated herein by reference), and may be used for species detection of the subject invention.

The species identification using DNA microarray comprises: obtaining a microarray containing templates derived from samples to be tested; obtaining MHV probe(s) which may be labeled; hybridizing the MHV-specific probe(s) to said microarray; and detecting the probes on said microarray.

The microarray format of the subject invention is particularly useful for simultaneous detection of multiple samples.

Microarray substrate and methods of attaching nucleic acid samples to a microarray substrate are well known in the art, for example, U.S. Pat. No. 5,807,522 (supra). The microarray according to the invention may comprise a plurality of DNA derived from multiple test sample.

Methods of generating and labeling probes for microarray analysis are well known in the art (U.S. Pat. Nos. 3,817,837; 3,850,752; 3,939,350; 3,996,345; 4,277,437; 4,275,149; and 4,366,241).

A probe according to the invention is 15 to 500 nucleotides, more preferably 30-200 nucleotides and most preferably 50 to 150 nucleotides in length. The probe may be single or double stranded, and may be a PCR fragment amplified from the appropriate conserved region of MHV.

Kits

The invention is intended to provide novel compositions and methods for the identification of Mouse Hepatitis virus in a test sample, as described herein. The invention herein also contemplates a kit format that comprises a package unit having one or more containers of the subject composition and in some embodiments including containers of various reagents used for polynucleotide synthesis, including synthesis in PCR. The kit may also contain one or more of the following items: polymerization enzymes, dNTPs, primers, buffers, instructions, and controls. The Kits may include containers of reagents mixed together in suitable proportions for performing the methods in accordance with the invention. Reagent containers preferably contain reagents in unit quantities that obviate measuring steps when performing the subject methods. In one embodiment, the kit contains one or more MHV-specific primers and probes which are complementary to a conserved region of the MHV genome.

In a preferred embodiment, the kit comprises a primer with SEQ ID NO: 2, a primer with SEQ ID NO: 3, and a probe with SEQ ID NO: 4.

D. Uses of the Invention

Current Methods of Monitoring Mouse Colonies for MHV

MHV is highly transmissible. The standard method for disease surveillance utilizes immunocompetent sentinel mice. A sentinel cage is dedicated to a subset of monitored cages. With each bedding change soiled bedding is transferred from the monitored group to the sentinel cage. Transmission of MHV is thought to be through fecal excretion and introduced into the naive recipient via ingestion and inhalation (Huang D S, Emancipator S N, Fletcher D R, Lamm M E, Mazanec M B. Hepatic pathology resulting from mouse hepatitis virus S in infection in severe combined immunodeficiency mice. Lab Anim Sci 1996; 46(2):167-73). It requires one to two weeks for detectable levels of antibody to be generated after exposure. MHV antibody titers start to increase on day six after experimental inoculation with MHV and peak at approximately day twenty (Yamada Y K, Yabe M, Yamada A, Taguchi F. Detection of mouse hepatitis virus by the polymerase chain reaction and its application to the rapid diagnosis of infection. Lab Anim Sci 1993; 43(4):285-90). Weekly serological screening for MHV would be optimal. However, this frequency is cost prohibitive for most institutions. New animals introduced into a colony from breeders are assumed to by negative for MHV and are not routinely screened.

Disease Monitoring for MHV

Sentinels are immunocompetent mice with a normal complement of T and B cells. These mice mount an immune response to MHV and clear the virus within 20 to 24 days after exposure. The use of an immunodeficient strain (SCID) as sentinels circumvents this clearance. However, 10 to 20 percent of SCID mice are leaky and may develop antibody mediated resistance (Bosma M, Schulet W, Bosma G. The SCID mouse mutant. Curr Top Microbiol Immunol 1998; 137:197-202). The use of RAG2 knockout mice (no mature T or B cells) as sentinels would be optimal. Each sentinel cage typically houses three Rag 2 and two immunocompetent mice. Three Rag 2 mice insure that at least one fecal sample will be obtained during each collection. All sentinel mice are maintained on a high fiber/low fat diet to foster fecal production. IFA or Elisa is used to confirm a MHV Molecular Beacon Assay positive finding utilizing the serum collected from the immunocompetent mouse. The immunocompetent mouse is also used to screen for other mouse pathogens in the colony.

Sentinel Exposure and Virus Containment

MHV can be detected as early as 24 hours in RNA from liver homogenates after inter-peritoneal injection (Yamada Y K, Yabe M, Yamada A, Taguchi F. Detection of mouse hepatitis virus by the polymerase chain reaction and its application to the rapid diagnosis of infection. Lab Anim Sci 1993; 43(4):285-90). Casebolt et al reported reliable detection of MHV in feces as early as 3 days post oronasal inoculation (Casebolt D B, Qian B, Stephensen C B. Detection of enterotropic mouse hepatitis virus fecal excretion by polymerase chain reaction. Lab Anim Sci 1997; 47(1):6-10).

Sentinel exposure is dependent on transfer of soiled bedding from monitored cages. This occurs on a weekly basis. As a baseline, it requires 3 days from exposure to detect MHV in fecal samples. Optimal sampling should be conducted on all sentinel cages 72 hours after cage changing. Results from the MHV Molecular Beacon Assay can easily be obtained within eight hours after samples are taken. Therefore, by day four post cage change any positive result will give ample time to quarantine and extensively test mice from the suspect group before the next cage change. In this way, the potential risk of spread of MHV is eliminated. Serological testing however typically requires one week from receipt of serum to obtain test data. This length of time results in another round of cage being performed before test results are obtained and therefore greatly increases the chance that any MHV infection could spread within the colony.

Screening Newly Introduced Animals for MHV

Often new animals are imported into a mouse colony from outside vendors for breeding. It usually requires one week for these mice to acclimate to the light cycle in the room and after one week they are competent to breed. These mice are assumed to be MHV free. However, there is still a potential risk of introduction of MHV into the colony. One RAG2 sentinel cage would be designated to screen these mice. Upon arrival the new mice will be quarantined and not exposed to the main colony. One mouse from the new shipment would be co-housed with the RAG2 sentinel. After 3 days of exposure, a fecal sample would be collected from the sentinel cage and each cage from the new arrival group. By day 4 results from the MHV Molecular Beacon Assay can easily be obtained. If the test results are negative the mice will be introduced into the colony on schedule. If there is a positive result, the group will be quarantined and extensively tested to determine the extent of the contamination. This offers optimal protection to the main colony while not affecting productivity.

MHV Disease Time Course and Application of MHV Molecular Beacons Assay

Naïve mice develop detectable antibody titers at six days post exposure to MHV (15). Antibody titers increase and within 20-24 days the virus is cleared in most cases. MHV has a high mutation frequency and within a chronically infected colony new strains may develop and reinfect a previously immune animal.

After the first positive finding further serological testing of experimental animals would require introduction of new disease free sentinels. However, this is ineffective in determining if an active infection persists in a specific experimental animal. The MHV Molecular Beacons Assay can however determine if a new strain variant is present within a colony.

The standard procedure to eliminate a MHV contamination in a mouse colony is to rederive each sero-positive line and/or sacrifice all mice in the colony. Both of these alternatives are time consuming and expensive. Rederivation takes 10 weeks or longer to establish breeding age mice. Lines with multiple transgenic and/or gene targeted alleles can take several generations to reestablish an experimental cohort. This process can take months. Likewise, if one is forced to sacrifice experimental animals due to such factors as lack of fertility or old age. The only recourse is to generate a new transgenic or gene targeted lines. If a specific line has been well characterized and is in a low fertility strain background the process can take years.

Once a mouse is determined to be sero-positive for MHV it will remain so indefinitely. It is difficult to determine if an animal is actively shedding virus by current serological methods. The present invention can determine if virus is being shed in sero-positive mice. Thus, by screening valuable experimental animals by this method it becomes possible to circumvent the need for rederivation or sacrifice.

Screening for Disease Status in Sero-Positive Mice

Due to modern microisolotor cage technology it is rare that all mice within a sentinel positive group are MHV infected. If a sentinel animal for an experimental cohort is determined to be MHV positive, breeding is stopped, mice are quarantined, cages are not changed and mice are maintained in the same cages. After 72 hours each mouse is tested by the MHV Molecular Beacons Assay. Mice that test negative are presumed to have cleared the virus or were not exposed and are used to reestablish the experimental colony. Animals testing positive will continue to be quarantined. Based of their experimental value, mice can be retested after a period of 26 days. The mice will be expected to have cleared the virus by this time and can be reintroduced into the colony. In this way, the need for rederivation and/or sacrifice is eliminated.

### EXAMPLES

The Use of the MHV Molecular Beacons Assay to Detect the Time Course of MHV Infection in Laboratory Mice

Two chronically MHV infected ICR mice were co-housed with one SCID and one naïve immunocompetent mouse for twenty-four days. At the end of this period serum was collected from the immunocompetent mouse and screened by ELISA for MHV. The test indicated that the mouse was MHV positive and based on this result the SCID mouse was assumed to be MHV positive. At the zero time point, two naive SCID mice were co-housed with the presumed MHV positive SCID. Fresh fecal samples were obtained at 24, 48, 72 and 120 hours post exposure. Two experiments were conducted independently.

Total RNA from the fresh fecal samples was isolated using a Qiagen RNeasy Micro Isolation Kit Cat. # 74103 according to manufacturer's instructions. All samples were treated with DNAse to eliminate contaminating genomic DNA in the samples.

MHV-specific sequences, present in the fecal samples, are amplified by PCR using MHV specific primers. Addition of a MHV-specific molecular beacon probe to the PCR reaction mixture ensures accurate and sensitive detection of MHV sequences in the samples. The MHV-specific primer sequences used in this experiment are:

| | | |
|---|---|---|
| MHV31053f: | GGCGCTCGGTGGTAACC | (SEQ ID NO: 2) |
| MHV31125r: | TGACAGCAAGACATCCATTCTGA | (SEQ ID NO: 3) |

PCR amplication of MHV sequences using these primers is predicted to produce an amplicon of 73 nucleotides in length that corresponds to the conserved sequence of SEQ ID NO: 1 that starts at nucleotide 31043 and finishes at nucleotide 31150 of the MHV genomic sequence (numbering according to the MHV sequence of Genbank Accession Number: NC 001846).

The sequence of the MHV-specific molecular beacon probe used in this experiment is:

MHV31071MB:

(SEQ ID NO: 4)

FAM-GTGCCGGAGTGTCCTATCCCGACTTTCTCGCGAGCCGGCAC-QSY

The reference gene used for this experiment is the housekeeping gene, GAPDH. Both GAPDH primers and probes are commercially available from PE Applied Biosystems (GAPDH Control Primers and Probe: Cat. # P/N 402869).

The GADPH-specific primer sequences are:

| | | |
|---|---|---|
| GAPDH F | GAAGGTGAAGGTCGGAGTC | (SEQ ID NO: 5) |
| GAPDH R | GAAGATGGTTAGGGGATTC | (SEQ ID NO: 6) |

The GAPDH-specific molecular beacon probe sequence is:

| | |
|---|---|
| GAPDH Probe | |
| VIC-CAAGCTTCCCGTTCTCAGCC-TAMRA | (SEQ ID NO: 7) |

GAPDH is used in this assay as an internal control (reference gene) to test for RNA integrity, RT reaction conditions and PCR reaction conditions. However, primers probe to any housekeeping gene can be used for this purpose.

The following reactants are mixed and aliquoted into an optical quality 200 μL PCR tube:

| | | |
|---|---|---|
| Taq Buffer 10× | 2.5 | μL per reaction |
| MgCl2 25 mM | 5.5 | |
| RNA 100 ng/μl | 1.0 | |
| dNTP's 10 mM | 0.5 | |
| MHV30153f 10 μM | 0.75 | |
| MHV31125r 10 μM | 0.75 | |
| MHV31071MB 20 μM | 0.5 | |
| GAPDH F 10 μM | 0.75 | |
| GAPDH R 10 μM | 0.75 | |
| GAPDH P 5 μM | 0.5 | |
| MuLV RT 20 U/μL | 0.25 | |
| Taq 5 U/μL | 0.25 | |
| Control Rodent RNA 50 ng/μL | 2.0 | |
| DEPC H2O | 11.0 | |
| Total | 25 | μL |

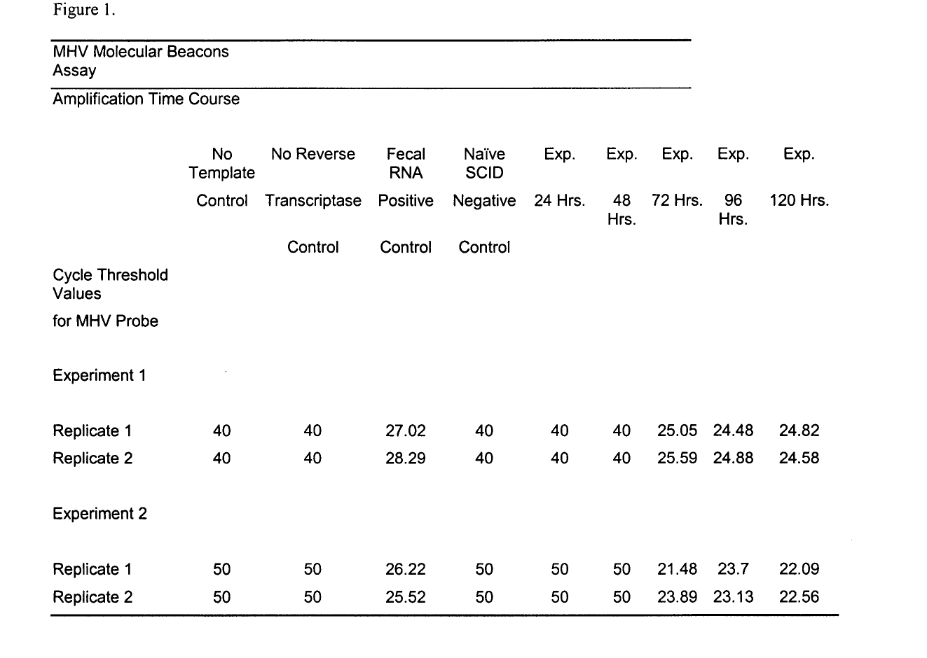

The tubes are then capped and placed into a real-time quantitative PCR instrument and cycled according to the following parameters:
1 Cycle of 48° C. for 60 min.
1 Cycle of 95° C. for 3 min.
40 Cycles of 95° C. for 15 sec.
40 Cycles of 60° C. for 30 sec.
   Fluorescent readings are taken during the last cycling step. The results of the experiment are reported in FIG. 1. The data indicates that at 72 hours post exposure, MHV is detected in the feces of the previously naïve SCID mouse.

1. An isolated conserved region of the Mouse Hepatitis virus genome encompassing at least 20 nucleotides of the DNA sequence consisting of SEQ ID NO: 1.

2. A synthetic polynucleotide primer consisting of the DNA sequence of SEQ ID NO:2.

3. A synthetic polynucleotide primer consisting of the DNA sequence of SEQ ID NO:3.

4. A polynucleotide probe consisting of the DNA sequence of SEQ ID NO: 4.

5. The probe according to claim 4, wherein said probe is a molecular beacon probe.

6. A kit encompassing a pair of primers consisting of the DNA sequences of SEQ ID NO: 2 and SEQ ID NO:3, and packaging materials therefore.

7. A kit according to claim 6, further encompassing a probe consisting of the DNA sequence consisting of SEQ ID NO: 4.

8. A kit encompassing a probe consisting of the DNA sequence of SEQ ID NO: 4, and packaging materials therefore.

9. The kit according to claims 6 or 8 further encompassing reverse transcriptase and a nucleic acid polymerase.

10. The kit of claim 9, wherein said nucleic acid polymerase is thermostable.

11. The kit according to claim 6 or 8 further encompassing a pair of reference gene-specific polynucleotide primers and a reference gene-specific probe.

12. The kit of claim 7, wherein said probe is a molecular beacon probe.

13. The kit of claim 8, wherein said probe is a molecular beacon probe.

14. A method to detect Mouse Hepatitis virus (MHV) nucleic acid in a sample encompassing reverse transcribing RNA of a mouse sample using MHV-specific polynucleotide primers to produce MHV-specific cDNA.

15. A method to detect Mouse Hepatitis virus (MHV) nucleic acid in a sample encompassing amplifying a conserved region of Mouse Hepatitis cDNA.

16. A method to detect Mouse Hepatitis virus using (MHV) nucleic acid in a sample encompassing:
a) reverse transcribing RNA of a mouse sample using MHV-specific polynucleotide primers to produce MHV-specific cDNA; and
b) amplifying a conserved region of said MHV-specific cDNA.

17. A method to detect Mouse Hepatitis virus (MHV) nucleic acid in a sample encompassing:
a) reverse transcribing RNA of a mouse sample using MHV-specific polynucleotide primers to produce MHV-specific cDNA;
b) amplifying a conserved region of said MHV-specific cDNA; and
c) detecting the presence of said conserved region using a MHV-specific probe.

18. A method to detect Mouse Hepatitis virus (MHV) nucleic acid in a plurality of mouse samples encompassing:
a) reverse transcribing RNA of each of a plurality of mouse samples using MHV-specific polynucleotide primers to produce a corresponding plurality of MHV-specific cDNAs;
b) amplifying a MHV conserved region of said MHV-specific cDNAs; and
c) detecting the presence of said conserved region using a MHV-specific probe.

19. The methods according to claims 14, 16 and 17, wherein said MHV-specific polynucleotide primers comprise the DNA sequence consisting of SEQ ID NO: 2 and SEQ ID NO:3.

20. The methods according to claims 14,15, 16, 17 and 18, wherein said polynucleotide primers are labeled.

21. The methods according to claims 15, 16, 17 and 18 wherein said conserved region comprises at least 20 nucleotides in the DNA sequence consisting of SEQ ID NO: 1.

22. The method according to claim 16 and 17, further encompassing the steps of:
a) reverse transcribing RNA of a mouse sample using reference gene-specific polynucleotide primers to produce reference gene-specific cDNA;
b) amplifying said reference gene-specific cDNA; and
c) detecting the presence of said reference gene-specific cDNA using a reference gene-specific probe.

23. The method according to claims 18, further encompassing the steps of:
a) reverse transcribing RNA of a plurality of mouse samples using reference gene-specific polynucleotide primers to produce a corresponding plurality of reference gene-specific cDNA;
b) amplifying said reference gene-specific cDNA; and
c) detecting the presence of said reference gene-specific cDNA using a reference gene-specific probe.

24. The method of claims 17, 18, 22 and 23, wherein said probe is a molecular beacon probe.


SEQUENCE LISTING (7 sequences)

SEQ ID NO: 1 - DNA, 108 bp, Murine hepatitis virus
atcctatgtcggcgctcggtggtaacccctcgcgagaaagtcgggataggacactctctatcagaatggatgtcttgctgtcataacagatagagaaggttgtggcag

SEQ ID NO: 2 - DNA, 17 bp, artificial sequence
  synthetic ligonucleotide primer
ggcgctcggtggtaacc

SEQ ID NO: 3 - DNA, 23 bp, artificial sequence
  synthetic oligonucleotide primer
tgacagcaagacatccattctga

SEQ ID NO: 4 - DNA, 41 bp, artificial sequence
  Synthetic molocular beacon probe
gtgccggagtgtcctatcccgactttctcgcgagccggcac

SEQ ID NO: 5 - DNA, 19 bp, artificial sequence
  Synthetic oligonucleotide primer
gaaggtgaaggtcggagtc

SEQ ID NO: 6 - DNA, 19 bp, artificial sequence
  syntheitic oligonucleotide primer
gaagatggttaggggattc

SEQ ID NO: 7 - DNA, 20 bp, artificial sequence
  Synthetic molocular beacon probe
caagcttcccgttctcagcc